SEQUENCE LISTING (27 sequences)

SEQ ID NO: 1 - DNA, 10 bp, Artificial Sequence
  oligo RNA
  misc_feature (1)..(4): n is a, c, g, t or u
  misc_feature (6)..(6): n is a, c, g, t or u
  misc_feature (8)..(10): n is a, c, g, t or u
nnnnancnnn

SEQ ID NO: 2 - RNA, 8 nt, Artificial Sequence
  oligo RNA
gucaucuu

SEQ ID NO: 3 - RNA, 8 nt, Artificial Sequence
  oligo RNA
gucaccuu

SEQ ID NO: 4 - RNA, 8 nt, Artificial Sequence
  oligo RNA
gccaucug

SEQ ID NO: 5 - RNA, 8 nt, Artificial Sequence
  oligo RNA
ugcauccu

SEQ ID NO: 6 - RNA, 8 nt, Artificial Sequence
  oligo RNA
accaucug

SEQ ID NO: 7 - RNA, 8 nt, Artificial Sequence
  oligo RNA
caccaucg

SEQ ID NO: 8 - RNA, 7 nt, Artificial Sequence
  oligo RNA
caccauc

SEQ ID NO: 9 - RNA, 6 nt, Artificial Sequence
  oligo RNA
ucaucu

SEQ ID NO: 10 - RNA, 6 nt, Artificial Sequence
  oligo RNA
ucaccu

SEQ ID NO: 11 - RNA, 6 nt, Artificial Sequence
  oligo RNA
ccaucu

SEQ ID NO: 12 - RNA, 6 nt, Artificial Sequence
  oligo RNA
gcaucc

SEQ ID NO: 13 - RNA, 6 nt, Artificial Sequence
  oligo RNA
ccaucu

SEQ ID NO: 14 - RNA, 6 nt, Artificial Sequence
  oligo RNA
accauc

SEQ ID NO: 15 - RNA, 5 nt, Artificial Sequence
  oligo RNA
uyauc

SEQ ID NO: 16 - RNA, 5 nt, Artificial Sequence
  oligo RNA
ucacc

SEQ ID NO: 17 - RNA, 4 nt, Artificial Sequence
  oligo RNA
cauc

SEQ ID NO: 18 - RNA, 4 nt, Artificial Sequence
  oligo RNA
cacc

SEQ ID NO: 19 - RNA, 4 nt, Artificial Sequence
  oligo RNA
gauc

SEQ ID NO: 20 - RNA, 3 nt, Artificial Sequence
  oligo RNA
auc

SEQ ID NO: 21 - RNA, 20 nt, Artificial Sequence
  oligo RNA
gucaucuugauagucaucuu

SEQ ID NO: 22 - DNA, 65 bp, Artificial Sequence
  shLuc for
gatcccgtacgcggaatacttcgattcaagagatcgaagtattccgcgtacgttttttgctagcg

SEQ ID NO: 23 - DNA, 65 bp, Artificial Sequence
  shLuc rev
aattcgctagcaaaaaacgtacgcggaatacttcgatctcttgaatcgaagtattccgcgtacgg

SEQ ID NO: 24 - DNA, 65 bp, Artificial Sequence
  shYBX1 for
gatccggtcatcgcaacgaaggttttcaagagaaaccttcgttgcgatgaccttttttgctagcg

SEQ ID NO: 25 - DNA, 65 bp, Artificial Sequence
  shYBX1 rev
aattcgctagcaaaaaaggtcatcgcaacgaaggtttctcttgaaaaccttcgttgcgatgaccg

SEQ ID NO: 26 - RNA, 8 nt, Artificial Sequence
  oligo RNA
gucgucuu

SEQ ID NO: 27 - RNA, 20 nt, Artificial Sequence
  oligo RNA
agaucucguucguauucuua

(19) United States
(12) Patent Application Publication
(10) Pub. No.: US 2024/0002855 A1
(43) Pub. Date: Jan. 4, 2024

(54) Nucleic Acid Molecule Binding to YB-1 Protein

(71) Applicant: Center for Excellence in Molecular Cell Science, Chinese Academy of Sciences, Shanghai (CN)

(72) Inventors: JingYi Hui, Shanghai (CN); JinZhu Wang, Shanghai (CN); Hong Zhu, Shanghai (CN)

(21) Appl. No.: 18/254,616

(22) PCT Filed: Nov. 26, 2021
(86) PCT No.: PCT/CN2021/133551

§ 371 Date: May 26, 2023

(30) Foreign Application Priority Data
Nov. 27, 2020 (CN) 202011357844.3

(51) Int. Cl. C12N 15/113; C07K 14/82; C07K 19/00; A61P 35/00

(52) U.S. Cl. CPC C12N 15/1135; C07K 14/82; C07K 19/00; A61P 35/00; C12N 2320/33; C12N 2310/321

(57) ABSTRACT

Provided are a nucleic acid molecule binding to an YB-1 protein and an application thereof in diagnosis, prevention and treatment of cancers. The nucleic acid molecule comprises a sequence selected from the group consisting of: (1) a nucleic acid sequence shown in N₁N₂N₃N₄AN₅CN₆N₇N₈ (SEQ ID NO: 1), wherein N₁ is C, G, U, or none, N₂ is A, G, U, or none, N₃ is C, G, U, or none, N₄ is C, G, U, or none, N₅ is C or U, and each of N₆, N₇ and N₈ is independently C, G, U, or none; and (2) a complementary sequence of (1).

### RELATED APPLICATIONS

This application is a National Stage Application under 35 U.S.C. 371 of expired PCT application PCT/CN2021/133551designating the United States and filed Nov. 26, 2021; which claims the benefit of CN application number 202011357844.3 and filed Nov. 27, 2020, each of which are hereby incorporated by reference in their entireties.

### SEQUENCE LISTING

The instant application contains a Sequence Listing which has been submitted electronically in XML format and is hereby incorporated by reference in its entirety. Said XML copy, created on May 25, 2023, is named “203773_sequence listing” and is 5 KB in size.

### TECHNICAL FIELD The subject invention relates to the field of biomedicine, and mainly relates to a nucleic acid molecule that binds to YB-1 protein and uses thereof.

### TECHNICAL BACKGROUND

Cancer is one of the major diseases that threat human health worldwide nowadays. A tumor is an abnormal growth of local tissue cells that can invade and spread to other parts of the body. The ten hallmarks of cancer include sustaining proliferative signaling, evading growth suppressors, resisting cell death, enabling replicative immortality, inducing angiogenesis, activating invasion and metastasis, avoiding immune destruction, tumor- promoting inflammation, deregulating cellular energetics, and genome instability and mutation. Despite the progress of science and technology has been made, and many countries have invested huge manpower and resources in the basic research and translational application of cancer biology, it still lacks effective treatment method for many tumors.

Glioma is a common primary brain tumor. Among gliomas, the most malignant one belongs to glioblastoma multiforme (GBM) with an average survival less than 15 months. The current main treatments are surgical resection followed by radiotherapy and chemotherapy. In recent years, some targeted drugs have been tested in clinical trials, but they only yielded limited improvement of patient outcome. Therefore, there is an urgent need to identify new therapeutic targets and anti-tumor drugs.

RNA binding proteins (RBPs) are a class of proteins that can bind single- or double-stranded RNA. They generally have a modular structure containing one or several RNA binding domains (RBDs) and other functional domains. The common RNA binding domains include RNA recognition motifs (RRM), KH domains, cold shock domains (CSD), double-stranded RNA binding domains (dsRBD), Zinc fingers, Piwi/Argonaute/Zwilli (PAZ) and etc. The functional domains of RNA binding proteins often possess the properties of enzymatic catalysis, formation of homomultimers and binding to other proteins. The modular structure of RNA binding proteins determines their specificity on the recognition of RNA targets and their important functions in the regulation of gene expression. Based on the feature that RNA binding proteins can recognize their cognate RNA motifs with high specificity and affinity, researchers have designed and synthesized oligonucleotides containing RNA binding motifs of the RNA binding proteins and used them to target RNA binding proteins, thereby interfering their functions in cells.

Y box binding protein (YB-1, gene name YBX1) is a major mRNA packaging protein enriched in many cells. It is a DNA/RNA binding protein carrying an evolutionarily conserved cold shock domain. YB-1 protein contains an amino terminal alanine/proline-rich region, a cold shock domain in the middle, and a carboxy terminal acidic/basic amino acid repeat domain. YB-1 was first identified as a transcription factor capable of binding CCAAT box (Y box) double-stranded DNA. Subsequent reports show that YB-1 is a multifunctional nucleic acid-binding protein that can control the expression of target genes by regulating DNA transcription, mRNA splicing and stability, and protein translation. YB-1 protein is mainly localized in the cytoplasm, but its expression is elevated in various tumor cells and its localization undergoes nuclear translocation. Some clinical results have shown that high expression of YB-1 in the nuclei of tumor cells, especially in breast cancer cells, is associated with drug resistance and poor survival in treatment. Some reports also demonstrate that not only its high expression in the nucleus, but also the total expression level of YB-1 in the cell is closely related to the malignancy and prognosis of tumors. YB-1 is considered as an oncogene, and in YB-1 transgenic mice, it causes abnormal proliferation of mammary epithelial cells, resulting in invasive mammary tumors. At the molecular level, it has been shown that YB-1 can regulate the transcription and translation of some genes related to cell growth, malignant transformation, drug resistance, epithelial-mesenchymal transition (EMT) and etc., thereby promoting tumor proliferation and metastasis. YB-1 is highly expressed in glioblastoma and promotes cell proliferation by inhibiting the biogenesis of miR-29b. Therefore, there is an urgent need to identify the RNA binding motif of YB-1 protein and to develop drugs that can inhibit the functions of YB-1 protein.

### SUMMARY OF INVENTION In a first aspect of the present invention, it provides an RNA oligonucleotide comprising a sequence selected from the group consisting of:

(1) a nucleotide sequence shown by N₁N₂N₃N₄AN₅CN₆N₇N₈ (SEQ ID NO: 1),
wherein, N₁ is C, G, U or null, N₂ is A, G, U or null, N₃ is C, G, U or null, N₄ is C, G, U or null, N₅ is C or U, and each of N_{6,} N₇ and N₈ is independently C, G, U or null, and
(2) a complementary sequence of (1).

In one or more embodiments, N₁ is C, G, U or null, N₂ is A, G, U or null, N₃ is C, G, U or none, N₄ is C, U or null, N₅ is C or U, and each of N₆, N₇ and N₈ is independently C, G, U or null.

In one or more embodiments, N₁ is C, U or null, N₂ is A, G or null, N₃ is C or null, N₄ is C, U or null, N₅ is C or U, N₆ is G, U or null, N₇ is C, G or null, and N₈ is C, G, U or null.

In one or more embodiments, N₁ is U or null, N₂ is G or null, N₃ is C or null, N₄ is C, U or null, N₅ is C or U, N₆ is U or null, N₇ is G or null, and N₈ is C or null.

In one or more embodiments, the RNA oligonucleotide comprises any one of the sequences selected from the group consisting of: GUCAUCUU, GUCACCUU, GCCAUCUG, UGCAUCCU, ACCAUCUG, CACCAUCG, CACCAUC, UCAUCU, UCACCU, CCAUCU, GCAUCC, CCAUCU, ACCAUC, UYAUC, UCACC, CAUC, CACC, GAUC, and AUC.

Preferably, the RNA oligonucleotide comprises a sequence selected from the group consisting of: GUCAUCUU, CACCAUC, CAUC, CACC, GAUC, and AUC.

In one or more embodiments, the RNA oligonucleotide is 3-50 nts, 3-40 nts, 3-30 bp, 3-20 nts, or 3-10 nts in length.

In one or more embodiments, the RNA oligonucleotide comprises two or more copies of the nucleotide sequences, which are optionally linked by a linker sequence. Preferably, said two or more of the nucleotide sequences are identical. In one or more embodiments, the linker comprises a polynucleotide having 1-10 nucleotides selected from the group consisting of A, U, G and C. Preferably, the linker is a polynucleotide consist of 1-8 of nucleotides selected from A, U, and G. In one or more embodiments, the linker is GAUA.

In one or more embodiments, the RNA oligonucleotide comprises a sequence of GUCAUCUU or GUCAUCUUGAUAGUCAUCUU.

In one or more embodiments, the RNA oligonucleotide is a single-stranded or double- stranded nucleic acid.

In one or more embodiments, the RNA oligonucleotide targets an RNA binding protein. Preferably, the protein is YB-1 protein.

In one or more embodiments, at least one nucleotide or all nucleotides of the RNA oligonucleotide are modified nucleotides.

In one or more embodiments, the RNA oligonucleotide inhibits the activity of YB-1 protein or decreases the amount of YB-1 protein.

In one or more embodiments, the nucleotide modification is selected from the group consisting of: 2′-O-methyl (2′-O—Me), 2′-O-methoxyethyl (2′-MOE), 5′-methyl, nucleotide analogue, and phosphorothioate on a phosphate group. These modifications can increase the stability of the RNA oligonucleotides.

In one or more embodiments, the nucleotide analogue is a nucleotide whose ribose is modified, wherein the 2′-OH group of the nucleotide is substituted with a group selected from the group consisting of: H, OR, R, halogen, SH, SR, NH_{2,} NHR, N(R)₂ and CN, wherein R is C₁-C₆ alkyl, alkenyl or alkynyl, and halogen is F, Cl, Br or I.

In the present invention, it also provides a DNA oligonucleotide with a sequence corresponding to the sequence of the RNA oligonucleotide described in the first aspect of the present invention. In one or more embodiments, the DNA oligonucleotide is a single-stranded or double-stranded nucleic acid.

In the present invention, it also provides a nucleic acid construct comprising the DNA oligonucleotide described in any one of the embodiments as described herein.

In one or more embodiments, the nucleic acid construct is a cloning vector, a recombinant vector or an expression vector.

In the present invention, it also provides a cell comprising the RNA oligonucleotide and/or the DNA oligonucleotide described in any one of the embodiments as described herein.

In the present invention, it also provides a protein-RNA complex comprising the RNA oligonucleotide described in any one of the embodiments as described herein and an RNA binding protein bound thereto. Preferably, the protein is YB-1 protein. In one or more embodiments, the binding is a non-covalent binding, via such as hydrogen bonding.

In the present invention, it also provides a pharmaceutical composition comprising the RNA oligonucleotide, or the protein-RNA complex described in any one of the embodiments as described herein and a pharmaceutically acceptable excipient.

In the present invention, it also provides a method for isolating or purifying a protein, comprising: incubating a labeled RNA oligonucleotide described in any one of the embodiments as described herein with a candidate protein, and isolating or purifying the protein binding to the RNA oligonucleotide using the label.

In one or more embodiments, the label is biotin and the isolating or purifying step comprises contacting the incubation mixture with a solid phase conjugated with streptavidin.

In the present invention, it also provides a method of screening a substance that interacts with the RNA oligonucleotide described in any one of the embodiments as described herein, comprising mixing the RNA oligonucleotide with a candidate substance and screening for the substance that causes an altered property of the mixture.

In one or more embodiments, the property is mobility or dissociation constant.

In one or more embodiments, the substance is a protein, preferably an RNA binding protein.

In the present invention, it also provides a kit for screening a substance that interacts with the RNA oligonucleotide described in any one of the embodiments as described herein, comprising the RNA oligonucleotide and a reagent for detecting the altered property of a mixture comprising the RNA oligonucleotide and said substance.

In one or more embodiments, the property is mobility or dissociation constant.

In one or more embodiments, the substance is a protein, preferably an RNA binding protein.

In one or more embodiments, the reagent is one or more reagents selected from the group consisting of heparin, polyacrylamide, binding buffer, and washing buffer.

In the present invention, it also provides an in vitro method for mediating specific alteration (or modification) of a target protein in cells, comprising the steps of:
(1) contacting the cells with the RNA oligonucleotide of any one of the embodiments as described herein under a condition that enables the specific alteration of a target protein, and
(2) mediating a specific alteration of the target protein by the RNA oligonucleotide, and directing the RNA oligonucleotide to a protein with which it interacts.

In one or more embodiments, the protein is an RNA binding protein, preferably YB-1 protein.

In one or more embodiments, the alteration of protein is inhibition of protein activity.

In one or more embodiments, the contacting comprises introducing the RNA oligonucleotide into cells which are capable of generating the specific alteration (or modification) of a target protein.

In one or more embodiments, the introducing comprises a delivery mediated by a nucleic acid construct.

The invention also provides an in vitro method for decreasing the activity or amount of YB-1 protein, regulating mRNA alternative splicing, regulating miRNA biogenesis, regulating DNA damage, regulating RNA transcription, regulating RNA degradation, regulating protein translation, or regulating non-coding RNA processing, wherein the method comprises the step of contacting the RNA oligonucleotide described in any one of the embodiments as described herein with an YB-1 protein.

In one or more embodiments, the regulation of mRNA alternative splicing or regulation of miRNA biogenesis is up-regulation of alternative exon splicing or down- regulation of miRNA biogenesis, and the miRNA is miR-29b or its precursor or derivative thereof, including but not limited to miR-29b-2, pri-miR-29b-2, and pre-miR-29b-2.

The present invention also provides a method for decreasing the activity or amount of YB -1 protein, regulating mRNA alternative splicing, regulating miRNA biogenesis, regulating DNA damage, regulating RNA transcription, regulating RNA degradation, regulating protein translation, regulating non-coding RNA processing, preventing or treating an YB -1 protein-related disease or disorder, inhibiting proliferation, malignant transformation or metastasis of tumor cells, improving drug resistance, or inhibiting epithelial-mesenchymal transition (EMT), wherein the method comprises administering the RNA oligonucleotide, the protein-RNA complex or the pharmaceutical composition described in any one of the embodiments as described herein to a subject in need thereof.

In one or more embodiments, the regulation of mRNA alternative splicing or regulation of miRNA biogenesis is up-regulation of alternative exon splicing or down-regulation of miRNA biogenesis, and the miRNA is miR-29b or its precursor or derivative thereof, including but not limited to miR-29b-2, pri-miR-29b-2, and pre-miR-29b-2.

In one or more embodiments, the YB-1 protein-related disease or disorder includes an YB-1 protein-related tumor, such as lung cancer, breast cancer, neurospoagioma, glioma, cervical cancer, liver cancer, head and neck cancer, prostate cancer, lymphoma, bladder cancer, pancreatic cancer, hepatobiliary carcinoma, colorectal cancer, or osteosarcoma.

The present invention also provides use of the RNA oligonucleotides or the protein-RNA complexes described herein in the manufacture of a medicament for decreasing the activity or amount of YB-1 protein, regulating mRNA alternative splicing, regulating miRNA biogenesis, regulating DNA damage, regulating RNA transcription, regulating RNA degradation, regulating protein translation, regulating non-coding RNA processing, preventing or treating an YB-1 protein-related disease or disorder, inhibiting proliferation, malignant transformation or metastasis of tumor cell, improving drug resistance, or inhibiting epithelial-mesenchymal transition (EMT).

In one or more embodiments, the regulation of mRNA alternative splicing or regulation of miRNA biogenesis is up-regulation of alternative exon splicing or down-regulation of miRNA biogenesis, and the miRNA is miR-29b or its precursor or derivative thereof, including but not limited to miR-29b-2, pri-miR-29b-2, and pre-miR-29b-2.

In one or more embodiments, the YB-1 protein-related disease or disorder includes an YB-1 protein-related tumor, such as lung cancer, breast cancer, neurospoagioma, glioma, cervical cancer, liver cancer, head and neck cancer, prostate cancer, lymphoma, bladder cancer, pancreatic cancer, hepatobiliary carcinoma, colorectal cancer, or osteosarcoma.

The present invention also provides use of the RNA oligonucleotides or the protein-RNA complexes described herein in the manufacture of a kit for detecting, diagnosing, prognosing or monitoring an YB-1 protein-related disease and/or disorder, such as diagnosing tumor malignancy and prognosis.

### BRIEF DESCRIPTION OF FIGURES

FIG. 1 shows the RNA binding sequence of YB-1 protein. A and B, the RNA binding specificity of YB-1 protein detected by gel shift assay. C, the dissociation constants for YB-1 protein and the indicated RNA sequences in A and B as determined by filter binding assay.

FIG. 2 shows the inhibition of cell proliferation of glioblastoma cells by oligonucleotides containing RNA binding motif for YB-1 protein. A and B, the proliferation of U251 (A) and U87-MG (B) cells without treatment or transfected with 200 nM or 500 nM control or YBX1-1 oligonucleotides, respectively, determined by MTT assay. C and D, the effects of 100 nM control, YBX1-1 or YBX1-2 oligonucleotides transfected into U251 (C) and U87-MG (D) cells on cell proliferation detected by MTT assay.

FIG. 3 shows the inhibition of cell proliferation of lung and breast cancer cells by oligonucleotides containing RNA binding motif for YB-1 protein. A and B, the proliferation of lung cancer A549 (A) and breast cancer MDA-MB-231 (B) cells without treatment or transfected with 200 nM control or YBX1-1 oligonucleotides, respectively, determined by MTT assay.

FIG. 4 shows the inhibition of cell proliferation of glioblastoma cells induced by oligonucleotides containing YB-1 RNA binding motif via targeting YB-1 protein. A and B, the effect on cell proliferation of the U251 (A) and U87-MG (B) cells with 500 nM control or YBX1-1 oligonucleotides transfected into the control (shLuc) or YB-1-specific knockdown cells (shYBX1), respectively, determined by MTT assay.

FIG. 5 shows the inhibition of in vivo tumorigenesis of transplanted cells in mice by oligonucleotides containing RNA binding motifs for YB-1 protein. A, in vivo bioluminescence images of mice 28 days after injection of U87-MG cells transfected with 200 nM scrambled oligonucleotides or YBX1-2 oligonucleotides, respectively; B, bioluminescence intensity of mice for the times indicated after injection of U87-MG cells transfected with scrambled oligonucleotides or YBX1-2 oligonucleotides, respectively; C, survival curves of mice injected with U87-MG cells transfected with scrambled oligonucleotides or YBX1-2 oligonucleotides, respectively.

### DETAILED DESCRIPTION

Unless specific definitions are provided, the nomenclature used to describe the procedures and techniques of analytical chemistry, synthetic organic chemistry, and medicinal and pharmaceutical chemistry herein are those well-known and commonly used in the art. Chemical synthesis and analysis use standard techniques.

The inventors determined the RNA-binding motif of YB-1 as CAUC or CACC in vitro through the systematic evolution of ligands by exponential enrichment (SELEX) method. The results of gel shift experiments show that the second residue A and the fourth residue C are important for YB-1 to recognize its motif. In addition, through individual-nucleotide resolution UV cross-linking and immunoprecipitation coupled RNA-Seq (iCLIP-Seq) technology, UYAUC was identified as YB-1 RNA-binding motif at a genome-wide level in cells, which is very similar to the binding motif identified by SELEX. The inventors' experimental results indicate that YB-1 achieves target-specific alteration (modification) by recognizing its RNA binding motif, and that inhibition of the activity of YB-1 regulates mRNA alternative splicing and miRNA biogenesis.

Nucleic Acid Molecule

The nucleic acid molecules provided by the present invention can be RNA oligonucleotides or DNA oligonucleotides. The RNA oligonucleotides targeting RNA binding proteins (e.g. YB-1 protein) of the present invention comprises a nucleotide sequence selected from the following sequences: (1) the nucleotide sequence shown by N₁N₂N₃N₄AN₅CN₆N₇N₈ (SEQ ID NO: 1), wherein N₁ is C, G , U or null, N₂ is A, G, U or null, N₃ is C, G, U or null, N₄ is C, G, U or null, N₅ is C or U, and each of N_{6,} N₇ and N₈ is independently C, G , U or null, and (2) a reverse complementary sequence of (1). Exemplarily, the RNA oligonucleotides comprise any one of the sequences selected from the group consisting of: GUCAUCUU, GUCACCUU, GCCAUCUG, UGCAUCCU, ACCAUCUG, CACCAUCG, CACCAUC, UCAUCU, UCACCU, CCAUCU, GCAUCC, CCAUCU, ACCAUC, UYAUC, UCACC, CAUC, CACC, GAUC, and AUC. Preferably, the RNA oligonucleotides comprise any one of the sequences selected from the group consisting of: GUCAUCUU, CACCAUC, CAUC, CACC, GAUC, and AUC. Typically, the RNA oligonucleotides are 3-50 nts in length. The RNA oligonucleotides are single-stranded or double-stranded nucleic acids. When RNA oligonucleotides are described, Y refers to C or U.

The RNA oligonucleotides described herein may also comprise two or more copies of the nucleotide sequences, optionally linked by linker sequence(s) among the copy of nucleotide sequences. Preferably, the sequences of nucleic acid copies are identical. The linker described herein can be any nucleotide sequence, as long as it does not interfere with the binding of the RNA oligonucleotide to the target protein. An exemplary linker is GAUA.

In embodiments of inhibiting the activity of a protein targeted by the RNA oligonucleotide, at least one nucleotide of the RNA oligonucleotide is modified to stabilize the RNA oligonucleotide. In the present disclosure, such modifications include 2′-O-Me modification, 2′-O-methoxyethyl modification, 5′-methylation modification, nucleotide analogue, etc. The nucleotide analogue may be a nucleotide whose ribose is modified, wherein the 2′-OH group of the nucleotide is substituted with a group selected from the group consisting of: H, OR, R, halogen, SH, SR, NH₂, NHR, N(R)₂ and CN, wherein R is C₁-C₆ alkyl, alkenyl or alkynyl, and halogen is F, Cl, Br or I.

The sequence of the DNA oligonucleotide of the present invention corresponds to the sequence of the above-mentioned RNA oligonucleotide, that is, U is replaced by T in the sequence of the above-mentioned RNA oligonucleotide. The DNA oligonucleotide is single-stranded or double-stranded nucleic acid.

Protein-RNA Complex The inventors have discovered that binding of the specific RNA oligonucleotides to an YB-1 protein formed a protein-RNA complex. Accordingly, the present invention also provides a protein-RNA complex comprising the RNA oligonucleotide described in any one of the embodiments as described herein and an YB -1 protein bound thereto.

Nucleic Acid Constructs and Cells The present invention also provides a nucleic acid construct comprising the DNA oligonucleotide described in any one of the embodiments as described herein. In certain embodiments, the nucleic acid construct is a vector. The oligonucleotide of the present invention can be cloned into various vectors, e.g., plasmids, phages, phage derivatives, animal viruses, and cosmids. The vector may be a cloning vector, an expression vector, or a homologous recombination vector. Cloning vectors can be used to provide coding sequences for RNA oligonucleotides of the present invention, such as DNA oligonucleotides containing the corresponding DNA sequences. Expression vectors can be provided in the form of viral vectors to cells. “Expression” as used herein includes the processes of transcribing DNA into RNA, as well as the process of translating RNA into protein. Expression of the present oligonucleotides is generally achieved by operably linking the oligonucleotides of the present invention to a promoter and incorporating the construct into an expression vector. A typical expression vector contains a transcriptional terminator, an initiation sequence and a promoter to regulate the expression of the desired oligonucleotides sequence. Viral vector technology is well known in the art and described, for example, in Sambrook et al. (2001, Molecular Cloning: A Laboratory Manual, Cold Spring Harbor Laboratory, New York) and other handbooks of virology and molecular biology. Viruses useful as vectors include, but are not limited to, retroviruses, adenoviruses, adeno-associated viruses, herpesviruses, and lentiviruses. Homologous recombination vectors are used to integrate the expression frame described herein into the host genome.

Mediating specific alteration of a target protein is accomplished by introducing a nucleic acid construct expressing the RNA oligonucleotide(s) described above into cells. The nucleic acid construct contains the sequences of the DNA oligonucleotide(s) described herein, and one or more regulatory sequences operably linked to these sequences. The DNA oligonucleotides described herein can be manipulated in a variety of ways to express the above-mentioned RNA oligonucleotides. The nucleic acid construct can be manipulated according to the difference of the expression vector or requirements prior to the insertion of the nucleic acid construct into the vector. Techniques that utilize recombinant DNA methods to change the nucleotide sequences are known in the art.

Regulatory sequences may comprise suitable promoter sequences. The promoter sequence is usually operably linked to the coding sequence of the RNA molecule or protein to be expressed. The promoter can be any nucleotide sequence that shows transcriptional activity in the host cell of choice, including mutated, truncated, and hybrid promoters, and can be derived from genes encoding extracellular or intracellular polypeptides homologous or heterologous to the host cell. The regulatory sequence may also comprise a suitable transcription termination sequence, which is recognized by a host cell to terminate transcription. A terminator sequence is operably linked to the 3′ terminal of the nucleotide sequence encoding the polypeptide. Any terminator that is functional in the host cell of choice may be used in the present invention. A leader sequence is operably linked to the 5′ terminal of the nucleotide sequence encoding the polypeptide.

To assess the expression of the RNA oligonucleotides in the present invention, the expression vector introduced into the cell may also contain either a label gene or a reporter gene or both to facilitate identification and selection of expressing cells from cells transfected or infected with viral vectors.

The DNA oligonucleotides described herein can generally be obtained by PCR amplification. Specifically, primers can be designed based on the nucleotide sequences disclosed herein, and the corresponding sequences can be amplified using nucleic acid constructs (e.g., vectors) containing DNA oligonucleotides described herein as templates. Alternatively, the DNA oligonucleotides described herein can be directly synthesized.

The RNA oligonucleotides described herein can be obtained by transcribing DNA into RNA. The transcription can take place intracellularly or extracellularly. Methods for DNA transcription in cells include introducing a nucleic acid construct (e.g., a vector) capable of directing DNA transcription into cells and incubating the cells under a condition for transcription. Methods for extracellular DNA transcription comprises: mixing a nucleic acid construct capable of directing DNA transcription (e.g., a vector) with RNA transcriptase and NTP and incubating under a condition for transcription. The reagents and methods required to perform the above transcription are known in the art. Alternatively, the RNA oligonucleotides described herein can also be directly synthesized.

Methods of introducing DNA or RNA into cells for gene expression are known in the art. Vectors can be introduced into host cells, e.g., mammalian, bacterial, yeast or insect cells, by any method known in the art. For example, expression vectors can be transfected into host cells by physical, chemical or biological methods. Physical methods of introducing polynucleotides into host cells include calcium phosphate precipitation, liposome transfection, particle bombardment, microinjection, electroporation, etc. Biological methods for introducing a polynucleotide of interest into host cells include the use of the vectors described above. Chemical methods of introducing polynucleotides into host cells include colloidal dispersion systems, such as macromolecular complexes, nanocapsules, microspheres, and beads; and lipid-based systems, including oil-in-water emulsions, micelles, mixed micelles, and liposome. Methods for direct transfection of RNA oligonucleotides into cells are well-known in the art, such as liposome transfection of RNA.

Host cells of the present disclosure contain and/or express the RNA oligonucleotides described in the present invention. When referring to cells containing or including, expressing a molecule such as an RNA oligonucleotide, the terms “containing” or “including” mean that the molecule is contained within the cell or on the cell surface; and term “expressing” means that the cells produce the molecule. Host cells include cells that ultimately express or contain the RNA oligonucleotides, also include various cells used in the production of these cells, such as E. coli cells, to provide the corresponding DNA sequences of the RNA oligonucleotides of the present invention or to provide the vectors described herein. In certain embodiments, provided herein are human brain glioblastoma cell lines U251 and U87-MG that stably express the RNA oligonucleotides described herein.

Pharmaceutical Composition and Administration The present invention also provides a pharmaceutical composition comprising a therapeutically effective amount of one or more the RNA oligonucleotides, the protein-RNA complexes of the present invention and a pharmaceutically acceptable excipient, such as diluent, carrier, solubilizer, emulsifier, preservative and/or adjuvant.

In certain embodiments, the acceptable diluents, carriers, solubilizers, emulsifiers, preservatives and/or adjuvants, etc. in the pharmaceutical composition are preferably nontoxic to recipients at the dosages and concentrations employed. In certain embodiments, the pharmaceutical composition may contain substances for improving, maintaining or preserving, for example, the pH, osmolarity, viscosity, clarity, color, isotonicity, odor, sterility, stability, the rate of dissolution or release, absorption or penetration of the composition. These substances are known from the prior art, e.g. see REMINGTON'S PHARMACEUTICAL SCIENCES, 18th Edition, A. R. Genrmo ed., 1990, Mack Publishing Company. Excipients suitable for delivering RNA oligonucleotides, protein-RNA complexes to target sites in vivo are well-known in the art. The optimal pharmaceutical composition may be determined by the intended route of administration, mode of delivery, and desired dosage.

The pharmaceutical compositions of the present invention may be selected for parenteral delivery. Alternatively, the composition may be selected for inhalation or delivery through the digestive tract, such as orally. The preparation of such pharmaceutically acceptable compositions is within the skill of the art.

Other pharmaceutical compositions suitable for RNA or RNA-protein complexes will be apparent to those skilled in the art, including formulations comprising RNA oligonucleotides in a sustained or controlled release delivery. Techniques for formulating a variety of other sustained or controlled release delivery formulations, such as liposomal vehicles, bioerodible microparticles or porous beads, and depot injections, are also known to those of skill in the art.

Pharmaceutical compositions for in vivo administration are usually provided in the form of sterile formulations. Sterilization is achieved by filtration through sterile filter membranes. When the composition is lyophilized, it can be sterilized using this method before or after lyophilization and reconstitution. Compositions for parenteral administration may be in lyophilized form or stored in solution. Parenteral compositions are usually presented in containers with sterile access pores, such as intravenous solution strips or vials with a stopper pierceable for a hypodermic needle.

Once formulated, the pharmaceutical compositions are stored in sterile vials as solutions, suspensions, gels, emulsions, solids, crystals, or as dehydrated or lyophilized powders. The formulations can be stored in ready-to-use form or reconstituted prior to administration (e.g., lyophilized). The present invention also provides kits for producing single-dose administration units. The kits of the present invention may each comprise a first container containing dried protein and a second container containing an aqueous formulation. In certain embodiments of the present invention, kits are provided containing single- compartment and multi-compartment pre-filled syringes (e.g., liquid syringes and lyophilized syringes).

The present invention also provides a method of treatment of a patient (especially an YB-1-related disease in a patient, such as an YB -1-related tumor) by administering the RNA oligonucleotide or the protein-RNA complex or a pharmaceutical composition thereof according to any embodiment of the present invention.

As used herein, the terms “patient”, “subject”, and “individual” are used interchangeably to include any organism, preferably an animal, more preferably a mammal (e.g., rat, mouse, dog, cats, rabbits, etc.), and most preferably humans. “Treatment” refers to the administration of a therapeutic regimen described herein to a subject to achieve at least one positive therapeutic effect (e.g., reduction in cancer cell number, reduction in tumor volume, reduction in the rate of cancer cell infiltration into surrounding organs, or reduction in the rate of tumor metastasis or tumor growth). Treatment regimens that effectively treat a patient can vary depending on factors such as the patient's disease state, age, weight, and the ability of the therapy to elicit an anticancer response in the subject.

“Effective amount” means an amount of a medicament sufficient to achieve the desired physiological result in an individual in need of the medicament. The therapeutically effective amount of a pharmaceutical composition containing an RNA oligonucleotide or protein-RNA complex of the present invention to be employed may depend on the molecule being delivered, the indication, the route of administration and the size of the patient (body weight, body surface or organ size) and/or status (age and general health), taxa of the individual to be treated, formulation of the composition, assessment of the individual's medical condition, and other relevant factors that vary from individual to individual. Those skilled in the art will appreciate that appropriate dosage levels for therapy will vary depending on the factors in part. In certain embodiments, the clinician can determine the dose and alter the route of administration to obtain optimal therapeutic effect.

“Dose” means a specified amount of an agent provided by a single administration or over a specified period of time. In certain embodiments, the dose may be administered in one, two or more boluses, tablets or injections. For example, in certain embodiments where subcutaneous administration is desired, the desired dose requires a volume that is not easily supplied by a single injection, and, therefore, two or more injections may be used to achieve the desired dose. In certain embodiments, the agent is administered by infusion, over an extended period of time or continuously. Dosages may be prescribed as hourly, daily, weekly or monthly doses.

The frequency of dosing will depend on the pharmacokinetic parameters of the specific RNA oligonucleotides or protein-RNA complexes in the formulation used. Clinicians typically administer the compositions until a dosage is reached that achieves the desired effect. The composition may thus be administered as a single dose, or as two or more doses over time (which may or may not contain the same amount of the desired molecule), or as a continuous infusion through an implanted device or catheter.

The pharmaceutical composition is administered by known methods, such as oral, intravenous, intraperitoneal, intracerebral (intraparenchymal), intracerebroventricular, intramuscular, intraocular, intraarterial, intraportal or intralesional route, or via sustained release systems or via implanted devices.

Diagnostic Uses, Assays, Screening and Kits

Based on the interaction of the RNA oligonucleotides described herein and RNA binding proteins (e.g. YB-1 protein), the present invention provides the following aspects.

RNA oligonucleotides of the present invention can be used in assays, e.g., binding assays, to detect and/or quantify YB-1 protein expressed in tissues or cells. The RNA oligonucleotides can be used in studies for further investigating the function of YB-1 protein in disease.

The RNA oligonucleotides of the present invention can be used for diagnostic purposes to detect, diagnose, prognose or monitor diseases and/or disorders associated with YB-1 protein, such as diagnosing tumor malignancy and prognosis. Detection of YB-1 protein can be performed in vivo or in vitro. Examples of methods suitable for detecting the presence or amount of YB-1 protein include gel shift assays, filter binding assays, SELEX, fluorescent staining, and the like. For diagnostic applications, RNA oligonucleotides are typically labeled with a detectable labeling group. Suitable labeling groups include: biotin, streptavidin and fluorophores. Various methods for labeling RNA oligonucleotides are known in the art and can be used in the present invention.

One aspect of the present invention provides methods of identifying cells expressing YB-1 protein. In a specific embodiment, the RNA oligonucleotide is labeled with a labeling group and the binding of the labeled RNA oligonucleotide to YB-1 protein is detected. In another specific embodiment, the binding of the RNA oligonucleotide to YB-1 protein is detected in vivo. In another specific embodiment, techniques known in the art are used to isolate and measure complexes composed of RNA oligonucleotides and YB-1 protein.

Another aspect of the present invention provides detection of the presence of a test molecule that competes with the RNA oligonucleotides of the description for binding to YB-1 protein. An example of one such assay involves detecting the amount of free RNA oligonucleotides in a solution containing an amount of YB-1 protein in the presence or absence of the test molecule. An increase in the amount of free RNA (i.e., RNA oligonucleotides that do not bind the YB -1 protein) will indicate that the test molecule is able to compete with the RNA oligonucleotide for binding to YB-1 protein. In one embodiment, the RNA oligonucleotide is labeled with a labeling group. Alternatively, the test molecule is labeled and the amount of free test molecule is monitored in the presence or absence of RNA oligonucleotides.

Yet another aspect of the present invention provides a method of isolating or purifying a protein, comprising: incubating the labeled RNA oligonucleotide described in any of the embodiments herein with a candidate protein, and isolating or purifying the protein binding to the RNA oligonucleotide using the label. The isolation or purification includes contacting the incubation mixture with a solid phase conjugated with a ligand capable of binding to the label. Exemplarily, suitable labels include biotin, and the isolation or purification includes contacting the incubation mixture with a solid phase conjugated with streptavidin. The present invention also provides a method and kit for screening for substances that interact with the RNA oligonucleotides described in any one of the embodiments as described herein. The method comprises mixing the RNA oligonucleotide with a candidate substance (e.g., RNA binding protein) and screening a substance that leads to property (e.g., mobility or dissociation constant) change of the mixture. The kit includes the RNA oligonucleotide and reagents for detecting changes in property of a mixture of the RNA oligonucleotide and a candidate substance (e.g., RNA binding protein). Depending on the property (e.g., mobility or dissociation constant) to be detected, the reagent may be selected from one or more of the following reagents: heparin, polyacrylamide, binding buffer, and washing buffer.

Method and Use The RNA oligonucleotides described herein can be used in an in vitro method for mediating alteration of a specific target protein (e.g., YB-1 protein) in cells, comprising: (1) contacting the cells with an RNA oligonucleotide according to any one of the embodiments as described herein under a condition that enables the specific alteration of the target protein, and (2) mediating the specific alteration by the RNA oligonucleotide, directing the RNA oligonucleotide to the protein with which it interacts. The specific alteration may be inhibition of protein activity. The contacting includes introducing (e.g., using a nucleic acid construct) the RNA oligonucleotide into cells in which specific alteration of a target protein can occur.

The RNA oligonucleotides described herein are capable of in vitro decreasing the activity or amount of YB-1 protein, regulating mRNA alternative splicing, regulating miRNA biogenesis, regulating DNA damage, regulating RNA transcription, regulating RNA degradation, regulating protein translation, regulating non-coding RNA processing, including the step of contacting the RNA oligonucleotide described in any of the embodiments herein with YB-1 protein. Regulation as described herein includes “up-regulation” or “down-regulation”. YB-1 protein is one of human mRNA packaging proteins, involved in the regulation of mRNA transcription, splicing, degradation, translation and other processes in the expression of protein-coding genes. The activities of YB-1 protein include the ability to bind RNA, regulate DNA damage, regulate RNA transcription, regulate splicing, regulate localization, regulate degradation, regulate translation, regulate non-coding RNA processing, etc.

The RNA oligonucleotides described herein can also be used in a method of in vivo reducing the activity or amount of YB-1 protein, regulating mRNA alternative splicing, regulating miRNA biogenesis, regulating DNA damage, regulating RNA transcription, regulating RNA degradation, regulating protein translation, regulating non-coding RNA processing, preventing or treating an YB-1 protein-related disease or disorder, inhibiting tumor (e.g. YB-1 protein-related tumor) cell proliferation, malignant transformation or metastasis, improving drug resistance, or inhibiting epithelial-mesenchymal transition (EMT). The method comprises administering the RNA oligonucleotide, the protein-RNA complex or the pharmaceutical composition as described in any one of the embodiments as described herein to an individual in need thereof. The YB-1 protein-related disease or disorder includes an YB-1 protein-related tumor, such as lung cancer, breast cancer, neurospoagioma, glioma, cervical cancer, liver cancer, head and neck cancer, prostate cancer, lymphoma, bladder cancer, pancreatic cancer, hepatobiliary cancer, colorectal cancer, or osteosarcoma, which are related to YB-1 protein.

In this regard, the RNA oligonucleotides or protein-RNA complexes described herein can be used in the manufacture of a medicament for decreasing the activity or amount of

YB-1 protein, regulating mRNA alternative splicing, regulating miRNA biogenesis, regulating DNA damage, regulating RNA transcription, regulating RNA degradation, regulating protein translation, regulating non-coding RNA processing, preventing or treating an YB-1 protein-related disease or disorder, inhibiting tumor cell proliferation, malignant transformation or metastasis, improving drug resistance, and inhibiting epithelial- mesenchymal transition (EMT).

The present invention will be illustrated by a way of specific examples below. It should be understood that these examples are merely explanatory and is not intended to limit the scope of the present invention. Unless otherwise specified, the methods and materials used in the examples are conventional materials and methods in the art.

### EXAMPLES

### Example 1: Materials and Methods

1.1 Gel Shift Assay

After ³² P-labeled RNA (130 fmol) was incubated with GST-YB -1 fusion protein in different molar ratios at 30° C., heparin was added to a final concentration of 5 mg/mL. The RNA-protein complexes were separated by 5% native polyacrylamide gel electrophoresis, and then detected by autoradiography.

1.2 Filter Binding Assay Filter binding assay was performed on a 96-well vacuum filtration device. ³²P-labeled RNAs (50 fmol) were incubated with different concentrations of GST-YB -1 protein for 20 min at room temperature in a total volume of 50 μL of binding buffer (final concentration of binding buffer: 10 mM Tris—Cl pH 8.0, 100 mM KCl, 2.5 mM MgCl₂, 0.01% NP40, 10% glycerol, and 0.2 mg/mL BSA). The reactants were added to a nitrocellulose membrane in a 96 well plate which was then washed 5 times with a wash buffer (10 mM Tris-Cl pH 8.0, 100 mM KCl, and 2.5 mM MgCl₂). After the membrane was dried, autoradiography was performed.

1.3 Cell Lines

Human glioma cell lines U251 and U87-MG were cultured with Dulbecco's modified Eagle's medium (DMEM) supplemented with 10% fetal bovine serum (GIBCO) in an incubator containing 5% CO₂ at 37° C.

1.4 shRNA Plasmid Construction

After mixing forward primer and reverse primer of the same moles for shLuc or shYBX1, the mixture was heated to 90° C. and annealed to room temperature. The annealed double-stranded DNA was ligated by a DNA ligase to a pSIREN-RetroQ vector that has undergone double enzyme digestion with EcoRI and BamHI, to obtain plasmids pSIREN-RetroQ-shLuc and pSIREN-RetroQ-shYBX1, respectively.

1.5 Establishment of Stable YB-1 Knockown Cell Lines

(1) 24 h before transfection, HEK293T cells were inoculated in a 6 cm culture dish, so that the density at the time of transfection was about 50%.

(2) Cells were transfected with 5 μg of viral plasmid pSIREN-RetroQ and 5 μg of packaging plasmid pHelper by a calcium phosphate method.

(3) The medium was changed 8-12 h after transfection and 3.5 mL of fresh medium was added.

(4) 48 h after transfection, a first medium containing virus particles was collected and stored at 4° C., and 3.5 mL of fresh medium was added at the same time.

(5) 72 h after transfection, a second medium containing virus particles was collected and combined with the first medium, to obtain totally about 6 mL of medium.

(6) 30 μL polybrene at a concentration of 4 mg/mL and 30 μL HEPES (pH 7.2) at a concentration of 20 mM were added, mixed well and filtered with 0.45 μm filter membrane.

(7) The filtered viruses were stored at 4° C. for a week or frozen at −80° C.

(8) 24 h before infection, U251 and U87-MG cells to be infected were inoculated in 6-well plates to allow the density before infection to reach 50%.

(9) 24 h after inoculating cells, the medium in the petri dish was removed, 2 mL of filtered viruses were added, the 6-well plate was placed in a horizontal centrifuge and centrifuged at 30° C., 2500 rpm for 90 min.

(10) After centrifugation, the viruses were discarded, the medium was replaced with a normal medium, and the cells were cultured in a cell incubator at 37° C. for 24 h.

(11) Appropriate concentration of puromycin was added to screen positive cells infected with virus (2 μg/mL for U251 cells, 1 μg/mL for U87-MG cells).

(12) Culturing with puromycin was maintained and the cells were passaged for 2-3 generations, and the knockdown efficiency of target protein YBX1 was detected.

(13) The stable knockdown cell lines were expanded and cryopreserved.

1.6 Establishment of U87-MG Cell Line Stably Expressing Luciferase

(1) 24 h before infection, U87 cells to be infected were inoculated in a 3.5 cm plate to allow the density before infection to reach 50%.

(2) 1 mL of luciferase-expressing virus (HanBio) was mixed with 1 mL of fresh medium containing 10%-fetal bovine serum in a ratio of 1:1 and added to U87 cells.

(3) 48 h after the viruses were added, puromycin was added to screen the positive cells expressing luciferase.

(4) After detecting cell fluorescence value and passaging the cells, the cells were frozen for subsequent experiments.

1.7 Oligonucleotides

| Name | Sequences |
|---|---|
| shLuc for | 5′-GATCCCGTACGCGGAATACTTCGATTCAAGAGATCG |
| | AAGTATTCCGCGTACGTTTTTTGCTAGCG-3′ |
| shLuc rev | 5′-AATTCGCTAGCAAAAAACGTACGCGGAATACTTCGA |
| | TCTCTTGAATCGAAGTATTCCGCGTACGG-3″ |
| shYBX1 for | 5′-GATCCGGTCATCGCAACGAAGGTTTTCAAGAGAAAC |
| | CTTCGTTGCGATGACCTTTTTTGCTAGCG-3′ |
| shYBX1 rev | 5′-AATTCGCTAGCAAAAAAGGTCATCGCAACGAAGGTT |
| | TCTCTTGAAAACCTTCGTTGCGATGACCG-3′ |
| Control | 5′-GUCGUCUU-3′, each base was modified |
| oligo | with 2-O-Me |
| Rearrange | 5′-AGAUCUCGUUCGUAUUCUUA-3′, each base |
| oligo | was modified with 2-O-Me |
| YBX1-1 | 5′-GUCAUCUU-3′, each base was modified |
| oligo | with 2-O-Me |
| YBX1-2 | 5′-GUCAUCUUGAUAGUCAUCUU-3′, each base |
| oligo | was modified with 2-O-Me |

1.8 MTT Experiment

(1) The cells were trypsinized into single cells and counted. Cells of different experimental groups were inoculated into a 24-well plate, with 2000 cells per well. Four duplicate wells were set for each group of cells. Cells were placed in a cell incubator for routine culture.

(2) After the cells adhered, the medium in the 24-well plate was discarded, 500 μL of freshly prepared MTT solution (5 mg/mL, dissolved in a serum-free medium) were added to each well, and the culture was continued in a 37° C. incubator for 4 hours.

(3) The medium supernatant in the wells was aspirated, and then 500 μL DMSO solution was added to each well. The plate was shaken slowly at room temperature for 10 min in the dark to fully dissolve the crystals.

(4) 200 μL of crystal solution was added to a 96-well plate for microplate reader detection.

(5) The absorption value of each well was measured on the microplate reader using light with a wavelength of 570 nm.

1.9 Tumorigenicity Experiment by Cell Transplantation

(1) The luciferase-expressing U87-MG cells were trypsinized into single cells and suspended in PBS. The cells were counted to make the concentration of cells to be 5×10⁵/μL. The injection volume of cell was 1 μL.

(2) The experimental mice were eight-week-old male NOD-SCID mice. The mice were weighed, and pentobarbital anesthetic was injected intraperitoneally for anesthesia according to the weight of the mice with 100 μL of 0.75% pentobarbital per 20 g. After anesthesia, the mice were placed on an operating table. The head of the mouse was disinfected with alcohol, the skin tissue of the mouse brain was cut open, the meninges were peeled off, the Bregma point of the mice was marked, and the injection electrode was adjusted to Bregma point, labeled as the origin (0, 0, 0). The injection electrode was fixed to the injection point of (0, 2, 2.75) in the left side of the mouse brain by adjusting the X, Y and Z coordinates of the stereotaxic apparatus. Then the injection site was drilled with a drill, and the cells were slowly injected with an electrode probe and stayed 2 minutes, and the wound was sutured. After the operation, the mouse was removed from the operating table and placed in the mouse cage to wake up.

(3) Different times were selected for in vivo mouse imaging. 200 μL of luciferase substrate (luciferin) in a concentration of 15 mg/mL was intraperitoneally injected.

Isoflurane was used for gas anesthesia, and 5 minutes later, the labeled mice were put into an in vivo imager for imaging.

(4) The fluorescence value of luciferase was calculated, plotted and analyzed.

### Example 2: RNA Sequence Bound by YB-1 Protein

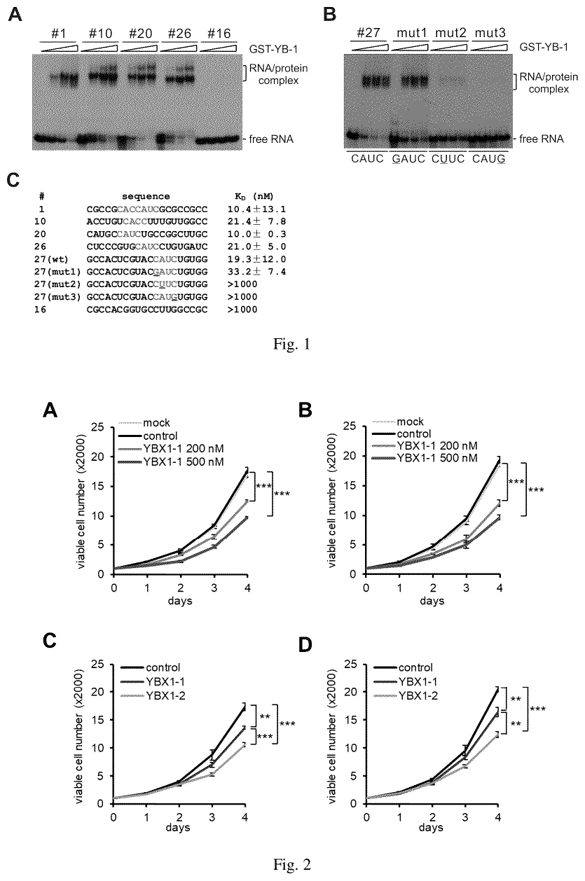

The RNA binding sequence of YB-1 protein was characterized by gel shift assay. The results show that YB-1 binds to sequences with CAUC or CACC. When the first C in the CAUC motif was mutated to G, YB-1 could still effectively bind to the GAUC sequence, indicating that YB-1 binding to RNA allows some flexibility. We also determined the dissociation constant of YB-1-RNA binding, which is at a level around nM, indicating that YB-1 binds to RNA with a high affinity. The change of a certain base in the RNA binding motif of YB-1 protein results in change with different amplitude of the dissociation constant. The results are shown in FIG. 1. Panels A and B show the RNA binding specificity of YB-1 protein detected by gel shift assay. After the ³² P-labeled RNA was incubated with 0, 2.5, 5 or 12.5 fold molar excess of GST-YB-1 fusion protein, the complexes formed by RNA and protein were separated by 5% native polyacrylamide gel electrophoresis, and results were observed by autoradiography. Panel C shows the dissociation constants for YB-1 protein and the indicated RNA sequences in panels A and B as determined by a filter binding assay.

### Example 3: Oligonucleotides Containing RNA Binding Motif of YB-1 Protein Inhibited the Proliferation of Glioma Cells

In order to detect the effect of oligonucleotides containing RNA-binding motifs of YB-1 protein on cell function, we first synthesized RNAs containing a CAUC 30 sequence and having a 2-O-Me modification at pentose of the nucleotide.

Oligonucleotides were then introduced into glioma cells U251 and U87-MG by liposome transfection of RNA, and cell proliferation experiments were carried out. With the MTT method, we found that cell proliferation was inhibited after cells were transfected with the RNAs having the CAUC sequence. However, transfection of RNAs with a CAUC mutation sequence, which was CGUC, did not affect cell proliferation. Furthermore, RNAs with two copies of the CAUC sequence had a stronger inhibition on cells than RNAs with one copy of CAUC. The results are shown in FIG. 2. Panels A and B show the cell proliferation of U251 (A) and U87-MG (B) without treatment (mock) or transfected with 200 nM or 500 nM of control or YBX1-1 oligonucleotides, respectively, as determined by MTT assay. Error bars represent standard errors of 4 sample replicates. Student's t-test: ***p<0.001. Panels C and D show the effects of U251 (C) and U87-MG (D) cells transfected with 100 nM control, YBX1-1 or YBX1-2 oligonucleotides on cell proliferation, as detected by MTT assay. Error bars represent standard errors of 4 sample replicates. Student's t-test: **p<0.01, ***p<0.001.

### Example 4: Oligonucleotides Containing RNA Binding Motif of YB-1 Inhibited Proliferation of Lung and Breast Cancer Cells

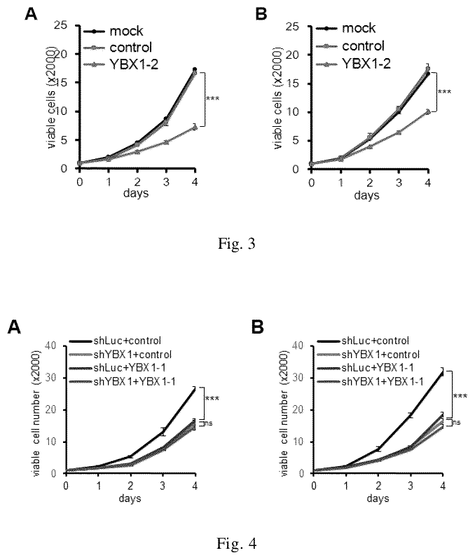

Since YB-1 protein is highly expressed in various tumors, we examined whether oligonucleotides containing RNA binding motifs of YB-1 protein could also inhibit proliferation of other tumor cells. When the oligonucleotides containing RNA binding motifs of YB-1 proteins were transfected into lung cancer cells A549 or breast cancer cells MDA-MB-231, similar to brain glioma cells, the oligonucleotides containing RNA binding motifs of YB-1 protein could also inhibit cell proliferation. This indicates that oligonucleotides containing RNA binding motifs of YB-1 protein have the potential to be effective in various tumor treatments. The results are shown in FIG. 3. Panels A and B show the cell proliferation of lung cancer cells A549 (A) and breast cancer cells MDA-MB-231 (B) without treatment or transfected with 200 nM control or YBX1-1 oligonucleotides, respectively, as detected by MTT assay. Error bars represent standard errors of 4 sample replicates. Student's t-test: ***p<0.001.

### Example 5: Oligonucleotides Containing RNA Binding Motif of YB-1 Protein Exerted Inhibitory Function on Cell Proliferation through Targeting YB-1 Protein

In order to determine that oligonucleotides containing RNA binding motifs of YB-1 protein could inhibit cell proliferation by targeting YB-1 protein, we used retrovirus to stably express shRNA that targeted YB-1 gene to establish YB-1 knockdown cell line. Compared with control cells, cell proliferation was inhibited when YB-1 was knocked down. However, when YB-1 knockdown cells were transfected with an RNA containing a CAUC sequence, cell proliferation was no longer affected by this RNA.

These results indicate that the oligonucleotides containing RNA binding motif of RNA binding protein exert the inhibitory function on cell proliferation by targeting YB-1 protein. The results are shown in FIG. 4. Panels A and B show the effect of 500 nM control and YBX1-1 oligonucleotides transfected into the control (shLuc) or the YB-1 knockdown cells (shYBX1), respectively, on cell proliferation of U251 (A) and U87-MG (B) cells, as determined by MTT assay. Error bars represent standard errors of 3 sample replicates. Student's t test: ns indicates no significant difference, ***p<0.001.

### Example 6: Oligonucleotides Containing RNA Binding Motif of YB-1 Protein Inhibited Tumorigenesis of Transplanted Cells in Mice In Vivo

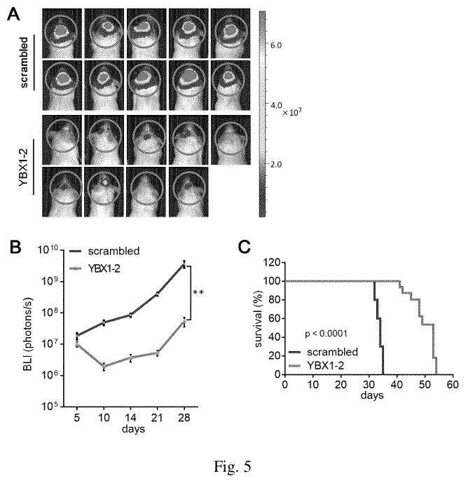

In order to determine the function of oligonucleotides containing RNA binding motifs of YB-1 protein on inhibiting tumor cell growth and malignant transformation in animals, we firstly constructed brain glioma U87 cells stably expressing luciferase. After transfected with RNAs having CAUC sequences, the cells were injected into the ventricles of mice. Through in vivo bioluminescence imaging in mice, we observed that the in vivo tumorigenic ability of cells transfected with RNAs having CAUC sequences was significantly reduced as compared with control cells, and the survival period was also significantly longer than that of control mice. The results are shown in FIG. 5. Panel A shows in vivo bioluminescence images of mice 28 days after injection of U87-MG cells transfected with 200 nM scrambled or YBX1-2 oligonucleotides, respectively, with the bioluminescence intensity indicating in photons/s/cm²/sr. Panel B shows a bioluminescence intensity (in photons/s) of mice for the times indicated after injection of U87-MG cells transfected with scrambled or YBX1-2 oligonucleotides, respectively. Error bars represent the standard error for each mouse in the two groups of samples. Mice injected with scrambled oligonucleotides: n=10; mice injected with YBX1-2 oligonucleotides: n=9. Student's t-test: **p<0.01. Panel C shows survival curves of mice injected with U87-MG cells transfected with scrambled oligonucleotides or YBX1-2 oligonucleotides, respectively.

The above examples demonstrate that oligonucleotides with RNA binding motifs of YB-1 protein have the function of inhibiting tumor cell proliferation and malignant transformation by specifically targeting YB-1 protein.

1. An RNA oligonucleotide targeting YB-1 protein, wherein the RNA oligonucleotide comprises a nucleotide sequence selected from the group consisting of:
(1) a nucleotide sequence shown by N₁N₂N₃N₄AN₅CN₆N₇N₈ (SEQ ID NO: 1), wherein N₁ is C, G, U or null, N₂ is A, G, U or null, N₃ is C, G, U or null, N₄ is C or G, N₅ is C or U, and each of N_{6,} N₇ and N₈ is independently C, G, U or null, and
(2) a complementary sequence of (1).

2. The RNA oligonucleotide according to claim 1, wherein:
the RNA oligonucleotide is 3-50 nts or 3-30 nts in length, or
N₁ is C, G, U or null, N₂ is A, G, U or null, N₃ is C, G, U or null, N₄ is C or G, N₅ is C or U, and each of N_{6,} N₇ and N₈ is independently C, G, U or null, or
N₁ is C, U or null, N₂ is A, G or null, N₃ is C or null, N₄ is C or G, N₅ is C or U, N₆ is G, U or null, N₇ is C, G or null, and N₈ is C, G, U or null, or
N₁ is U or null, N₂ is G or null, N₃ is C or null, N₄ is C or G, N₅ is C or U, N₆ is U or null, N₇ is G or null, and N₈ is C or null, or
the RNA oligonucleotide comprises two or more copies of the nucleotide sequences which are optionally linked by a linker sequence comprising 1-10 nucleotides selected from the group consisting of A, U, G, and C, or
at least one nucleotide in the RNA oligonucleotide is a modified nucleotide for stabilization of the oligonucleotide.

3. A protein-RNA complex comprising the RNA oligonucleotide according to claim 1 and an RNA binding protein bound thereto.

4. A pharmaceutical composition comprising the RNA oligonucleotide according to claim 1 and/or a protein-RNA complex comprising the RNA oligonucleotide and an RNA binding protein bound thereto, and a pharmaceutically acceptable excipient.

5. A method for isolating or purifying a protein, comprising: incubating a candidate protein with the RNA oligonucleotide according to claim 1 which is labeled, and isolating or purifying the protein bound to the RNA oligonucleotide using the label.

6. An in vitro method for mediating a specific alteration of a target protein in cells, comprising the steps of:
(1) contacting the cells with the RNA oligonucleotide according to claim 1, and
(2) mediating the specific alteration of a target protein by the RNA oligonucleotide, directing the RNA oligonucleotide to the protein with which it interacts.

7. A method for decreasing activity or amount of YB-1 protein, regulating mRNA alternative splicing, regulating miRNA biogenesis, regulating DNA damage, regulating RNA transcription, regulating RNA degradation, regulating protein translation, or regulating non-coding RNA processing, wherein the method comprises the step of contacting the RNA oligonucleotide according to claim 1 with the YB-1 protein in cells.

8-10. (canceled)

11. The RNA oligonucleotide according to claim 2, wherein:
the RNA oligonucleotide comprises a sequence selected from the group consisting of: GUCAUCUU, GUCACCUU, GCCAUCUG, UGCAUCCU, ACCAUCUG, CACCAUCG, CACCAUC, UCAUCU, UCACCU, CCAUCU, GCAUCC, CCAUCU, ACCAUC, UYAUC, UCACC, CAUC, CACC, GAUC, and AUC; and/or
the modification is selected from the group consisting of 2′-O-methyl, 2′-O-methoxyethyl, 5′-methyl, phosphorothioate and nucleotide analogues.

12. The protein-RNA complex according to claim 3, wherein:
the RNA binding protein is an YB-1 protein; and/or
the RNA oligonucleotides are 3-50 nts or 3-30 nts in length; and/or
at least one nucleotide in the RNA oligonucleotide is a modified nucleotide by for stabilization of the oligonucleotide; and/or
in SEQ ID NO:1, N₁ is C, G, U or null, N₂ is A, G, U or null, N₃ is C, G, U or null, N₄ is C or G, N₅ is C or U, and each of N_{6,} N₇ and N₈ is independently C, G, U or null; or N₁ is C, U or null, N₂ is A, G or null, N₃ is C or null, N₄ is C or G, N₅ is C or U, N₆ is G, U or null, N₇ is C, G or null, and N₈ is C, G, U or null; or N₁ is U or null, N₂ is G or null, N₃ is C or null, N₄ is C or G, N₅ is C or U, N₆ is U or null, N₇ is G or null, and N₈ is C or null; and/or
the RNA oligonucleotide comprises two or more copies of the nucleotide sequences which are optionally separated by a linker sequence comprising 1-10 nucleotides selected from the group consisting of A, U, G, and C.

13. The protein-RNA complex according to claim 12, wherein:
the RNA oligonucleotide comprises a sequence selected from the group consisting of: GUCAUCUU, GUCACCUU, GCCAUCUG, UGCAUCCU, ACCAUCUG, CACCAUCG, CACCAUC, UCAUCU, UCACCU, CCAUCU, GCAUCC, CCAUCU, ACCAUC, UYAUC, UCACC, CAUC, CACC, GAUC, and AUC; and/or
the modification is selected from the group consisting of 2′-O-methyl, 2′-O-methoxyethyl, 5′-methyl, phosphorothioate and nucleotide analogues.

14. The pharmaceutical composition according to claim 4, wherein:
the RNA binding protein is an YB-1 protein; and/or
the RNA oligonucleotides are 3-50 nts or 3-30 nts in length; and/or
at least one nucleotide in the RNA oligonucleotide is a modified nucleotide by for stabilization of the oligonucleotide; and/or
in SEQ ID NO:1, N₁ is C, G, U or null, N₂ is A, G, U or null, N₃ is C, G, U or null, N₄ is C or G, N₅ is C or U, and each of N_{6,} N₇ and N₈ is independently C, G, U or null; or N₁ is C, U or null, N₂ is A, G or null, N₃ is C or null, N₄ is C or G, N₅ is C or U, N₆ is G, U or null, N₇ is C, G or null, and N₈ is C, G, U or null; or N₁ is U or null, N₂ is G or null, N₃ is C or null, N₄ is C or G, N₅ is C or U, N₆ is U or null, N₇ is G or null, and N₈ is C or null; and/or
the RNA oligonucleotide comprises two or more copies of the nucleotide sequences which are optionally separated by a linker sequence comprising 1-10 nucleotides selected from the group consisting of A, U, G, and C.

15. The pharmaceutical composition according to claim 14, wherein:
the RNA oligonucleotide comprises a sequence selected from the group consisting of:
GUCAUCUU, GUCACCUU, GCCAUCUG, UGCAUCCU, ACCAUCUG, CACCAUCG, CACCAUC, UCAUCU, UCACCU, CCAUCU, GCAUCC, CCAUCU, ACCAUC, UYAUC, UCACC, CAUC, CACC, GAUC, and AUC; and/or
the modification is selected from the group consisting of 2′-O-methyl, 2′-O-methoxyethyl, 5′-methyl, phosphorothioate and nucleotide analogues.

16. The method according to claim 5, wherein the label is biotin and the isolating or purifying comprises contacting the incubation mixture with a solid phase conjugated with streptavidin.

17. The method according to claim 5, wherein:
the RNA binding protein is a YB-1 protein; and/or
the RNA oligonucleotides are 3-50 nts or 3-30 nts in length; and/or
at least one nucleotide in the RNA oligonucleotide is a modified nucleotide by for stabilization of the oligonucleotide; and/or
in SEQ ID NO:1, N₁ is C, G, U or null, N₂ is A, G, U or null, N₃ is C, G, U or null, N₄ is C or G, N₅ is C or U, and each of N_{6,} N₇ and N₈ is independently C, G, U or null; or N₁ is C, U or null, N₂ is A, G or null, N₃ is C or null, N₄ is C or G, N₅ is C or U, N₆ is G, U or null, N₇ is C, G or null, and N₈ is C, G, U or null; or N₁ is U or null, N₂ is G or null, N₃ is C or null, N₄ is C or G, N₅ is C or U, N₆ is U or null, N₇ is G or null, and N₈ is C or null; and/or
the RNA oligonucleotide comprises two or more copies of the nucleotide sequences which are optionally separated by a linker sequence comprising 1-10 nucleotides selected from the group consisting of A, U, G, and C.

18. The method according to claim 17, wherein:
the RNA oligonucleotide comprises a sequence selected from the group consisting of:
GUCAUCUU, GUCACCUU, GCCAUCUG, UGCAUCCU, ACCAUCUG, CACCAUCG, CACCAUC, UCAUCU, UCACCU, CCAUCU, GCAUCC, CCAUCU, ACCAUC, UYAUC, UCACC, CAUC, CACC, GAUC, and AUC; and/or
the modification is selected from the group consisting of 2′-O-methyl, 2′-O-methoxyethyl, 5′-methyl, nucleotide analogue and phosphorothioate.

19. The method according to claim 6, wherein:
the protein is an RNA binding protein, and/or
the protein alteration is inhibition of protein activity, and/or
the contacting includes introducing the RNA oligonucleotide into cells which are capable of generating the specific alteration of a target protein; and/or
the RNA binding protein is a YB-1 protein; and/or
the RNA oligonucleotides are 3-50 nts or 3-30 nts in length; and/or
at least one nucleotide in the RNA oligonucleotide is a modified nucleotide by for stabilization of the oligonucleotide; and/or
the RNA oligonucleotide comprises a sequence selected from the group consisting of: GUCAUCUU, GUCACCUU, GCCAUCUG, UGCAUCCU, ACCAUCUG, CACCAUCG, CACCAUC, UCAUCU, UCACCU, CCAUCU, GCAUCC, CCAUCU, ACCAUC, UYAUC, UCACC, CAUC, CACC, GAUC, and AUC.

20. The method according to claim 7, wherein:
the protein is an RNA binding protein, and/or
the protein alteration is inhibition of protein activity, and/or
the contacting includes introducing the RNA oligonucleotide into cells which are capable of generating the specific alteration of a target protein; and/or
the RNA binding protein is a YB-1 protein; and/or
the RNA oligonucleotides are 3-50 nts or 3-30 nts in length; and/or at least one nucleotide in the RNA oligonucleotide is a modified nucleotide by for stabilization of the oligonucleotide; and/or
the RNA oligonucleotide comprises a sequence selected from the group consisting of: GUCAUCUU, GUCACCUU, GCCAUCUG, UGCAUCCU, ACCAUCUG, CACCAUCG, CACCAUC, UCAUCU, UCACCU, CCAUCU, GCAUCC, CCAUCU, ACCAUC, UYAUC, UCACC, CAUC, CACC, GAUC, and AUC; and/or
the YB-1 protein-related disease or disorder comprises a YB-1 protein-related tumor

21. A method for preventing or treating a YB-1 protein-related disease or disorder, inhibiting tumor cell proliferation, malignant transformation or metastasis, improving drug resistance, or inhibiting epithelial-mesenchymal transition (EMT), comprising administering to a subject in need thereof an effective amount of the RNA oligonucleotide according to claim 1 and/or a protein-RNA complex comprising the RNA oligonucleotide and an RNA binding protein bound thereto, or a pharmaceutical composition comprising the RNA oligonucleotide and/or the protein-RNA complex.

22. The method according to claim 21, wherein the YB-1 protein-related disease or disorder comprises a YB-1 protein-related tumor.

23. The method according to claim 22, wherein the YB-1 protein-related tumor is lung cancer, breast cancer, neurospoagioma, glioma, cervical cancer, liver cancer, head and neck cancer, prostate cancer, lymphoma, bladder cancer, pancreatic cancer, hepatobiliary cancer, colorectal cancer, or osteosarcoma.
